(19) United States
(12) Patent Application Publication
(10) Pub. No.: US 2022/0117875 A1
(43) Pub. Date: Apr. 21, 2022

(54) COSMETIC COMPOSITIONS COMPRISING A CYCLODEXTRIN HAVING A PARTICLE SIZE DISTRIBUTION

(71) Applicant: ROQUETTE FRERES, Lestrem (FR)

(72) Inventors: Léon MENTINK, Lille (FR); Sophie PIOT, Paris (FR)

(21) Appl. No.: 17/309,552

(22) PCT Filed: Dec. 4, 2019
(86) PCT No.: PCT/FR2019/052932

§ 371 Date: Jun. 4, 2021

(30) Foreign Application Priority Data
Dec. 5, 2018 (FR) 18 72343
Nov. 5, 2019 (FR) 19 12367

(51) Int. Cl. A61K 8/73; A61K 8/06; A61K 8/92; A61K 8/34; A61K 8/37; A61Q 19/00

(52) U.S. Cl. CPC A61K 8/738; A61K 8/062; A61K 8/922; A61K 2800/412; A61K 8/375; A61K 8/73; A61Q 19/004; A61K 8/345

(57) ABSTRACT

Cosmetic compositions comprising at least one cyclodextrin which is at least partially present in the form of solid particles dispersed in a physiologically acceptable medium, that the cyclodextrin particles have a volume average size d(4,3), of less than or equal to 20 μm, more preferentially less than or equal to 12 μm, and most preferentially less than or equal to 8 μm, and a size distribution by volume, of which the characteristic diameters d(10), d(50) and d(90) are such that: a. the diameter d(10) is less than or equal to 5.0 μm, preferentially less than or equal to 2.5 μm, b. and the diameter d(50) is less than or equal to 15.0 μm, preferentially less than or equal to 10.0 μm, c. and the diameter d(90) is less than or equal to 30.0 μm, preferentially less than or equal to 25.0 μm.

### FIELD OF THE INVENTION

The invention relates to cosmetic or dermatological compositions comprising at least one cyclodextrin which is dispersed in the form of solid particles, said solid particles having a volume average size d(4.3), measured by laser particle size analysis, of less than or equal to 20 μm and a size distribution by volume, measured by laser diffraction particle size analysis, of which the characteristic diameters d(10), d(50) and d(90) are such that the diameter d(10) is less than or equal to 5.0 μm, the diameter d(50) is less than or equal to 15.0 μm, and the diameter d(90) is less than or equal to 30.0 μm. The cyclodextrin is thereby present in an amount greater than the solubility thereof in the physiologically acceptable medium of these compositions. Such compositions have an improved sensory profile, in particular regarding feel. The invention also relates to the use of said cyclodextrin in order to improve the sensory profile of cosmetic compositions, and as a sensory powder in cosmetics.

The cosmetic compositions according to the invention are able to be used in all fields of cosmetics, in particular in skin care and protection, bodily hygiene, hair care, oral-dental care, make-up and perfumes.

### BACKGROUND ART

A cosmetic product has to meet the needs of consumers, who demand efficacy, safety and sensory properties at the same time. Sensory perceptions form the main point of attraction of the consumer to the product used, well before benefits can be observed. Therefore, a cosmetic product must rise to the challenge of providing a beneficial cosmetic effect while providing as pleasant sensations as possible before or during application. In order to obtain a beneficial and attractive sensory profile, the formulations of cosmetic products have become increasingly complex over time by incorporating increasing amounts of functional or sensory ingredients of synthetic origin, generally originating from petrochemicals.

However, nowadays cosmetic products must respond to a new expectation of consumers: the natural origin, and, even more demandingly, the naturalness, of compositions. Indeed, consumers are now seeking cosmetic products composed substantially of natural ingredients or ingredients of natural origin, having as few chemical modifications or synthetic add-ons or petrochemical add-ons as possible.

The removal of functional or sensory ingredients originating from petrochemicals, or their substitution by ingredients of natural origin, constitutes an important direction for developing novel cosmetic products. However, the introduction of these novel natural ingredients, or ingredients of natural origin, may be accompanied by a more or less pronounced degradation of the sensory properties of the cosmetic product, in terms of their appearance, their handling, their application or their properties once applied to the skin or integuments. Thus, compositions meeting criteria of naturalness may prove difficult to spread, may pill, or may give, on application, a squeaky or brittle sensation, or else a sensation of not having sufficient glide. These inadequate or degraded sensory perceptions are detrimental to the quality or the image of a cosmetic product.

In the field of creams for skincare, for example, in particular those in the form of oil-in-water emulsions, the commonly used functional ingredients are emulsifiers which are surfactants originating from petrochemicals. The substitution of these surfactants with natural compounds is still an active area of research. A type of emulsifying system has been developed by the applicant and is the subject of a patent application FR1,762,841, not published to date. This emulsifying system is a mixture of a beta-cyclodextrin and a natural oil-in-water emulsifier, for example polyglyceryl-3-diisostearate, which makes it possible to adjust the physicochemical and sensory properties of the compositions as well as possible and to obtain oil-in-water emulsions with adapted and stable viscosity.

By producing emulsions, for example creams, with this natural emulsifying system developed by the applicant, very appealing and acceptable sensory perceptions were observed, but these perceptions could definitely be further perfected. Compared to formulations comprising petrochemical emulsifiers, the creams comprising this natural emulsifying system may, on some skins, be less easy to spread, penetrate less well, be liable to pill, and may have a less soft and less powdery, and sometimes more greasy, feel.

Sensory ingredients are ingredients added to the cosmetic composition in order to adjust the sensory profile thereof through physical effects. Among the most commonly used, there is talc, generally used to provide a soft, powdery and velvety feel, while enabling absorption of lipids, especially those of natural origin such as the sebum produced by the skin, or those introduced intentionally into a cosmetic composition. However, recent studies have indicated a possible toxicity of talc in the dry powder state, which is forcing the cosmetics industry to seek natural alternatives to this ingredient. For the sake of efficiency, a substitute is in particular sought which will reproduce the sensory perception when feeling talc, and its capacity to absorb lipids.

### BRIEF DESCRIPTION OF THE INVENTION

According to a first aspect, a subject of the present invention is a cosmetic or dermatological composition comprising at least one cyclodextrin which is at least partially present in the form of solid particles dispersed in a physiologically acceptable medium, said particles having a volume average size d(4.3), measured by laser particle size analysis, of less than or equal to 20 μm, preferentially less than or equal to 12 μm, and most preferentially less than or equal to 8 μm, and a size distribution by volume, measured by laser diffraction particle size analysis, of which the characteristic diameters d(10), d(50) and d(90) are such that:
a. the diameter d(10) is less than or equal to 5.0 μm, preferentially less than or equal to 2.5 μm,
b. the diameter d(50) is less than or equal to 15.0 μm, preferentially less than or equal to 10.0 μm, and
c. the diameter d(90) is less than or equal to 30.0 μm, preferentially less than or equal to 25.0 μm.
The invention also proposes a solution for improving the sensory properties of cosmetic products by means of such a cyclodextrin.

Indeed, the applicant discovered, surprisingly, that the use of a cyclodextrin in the form of a powder, composed of particles of a selected size, and preferably selected based on their particular size distribution, made it possible to obtain cosmetic compositions having highly advantageous and improved sensory properties, in particular in terms of ease of spreading and of soft, powdery, non-greasy feel.

In cosmetic compositions, cyclodextrins are usually used as a stabilizing agent for active molecules, or as an agent for improving the bioavailability of active molecules, or else as an agent promoting the dispersibility or water-solubility of active molecules. Cyclodextrins are thus known to improve the efficacy of cosmetic active ingredients, or even to make it possible to observe the effects thereof. However, cyclodextrins are not known to improve the sensory properties of cosmetic or dermatological compositions.

For the purposes of the present invention, cosmetic or dermatological composition or product is understood to mean any mixture of raw materials intended to be brought into contact with keratin materials (such as the skin, hair, body hair, nails, lips and external genitalia, or with the teeth and oral mucous membranes, with a view, exclusively or predominantly, to cleaning them, fragrancing them, modifying the appearance thereof, protecting them, keeping them in a good state or fixing body odors. This definition also includes dermocosmetic compositions. However, it excludes sanitary items such as maxi pads or diapers.

A second subject of the invention relates to the use, in a cosmetic or dermatological composition, of at least one cyclodextrin which is at least partially present in the form of solid particles dispersed in a physiologically acceptable medium, the cyclodextrin particles have a volume average size d(4.3), measured by laser particle size analysis, of less than or equal to 20 μm, more preferentially less than or equal to 12 μm, and most preferentially less than or equal to 8 μm, and a size distribution by volume, measured by laser diffraction particle size analysis, of which the characteristic diameters d(10), d(50) and d(90) are such that:
a. the diameter d(10) is less than or equal to 5.0 μm, preferentially less than or equal to 2.5 μm,
b. the diameter d(50) is less than or equal to 15.0 μm, preferentially less than or equal to 10.0 μm, and
c. and the diameter d(90) is less than or equal to 30.0 μm, preferentially less than or equal to 25.0 μm,
for improving the sensory properties of said composition, preferentially for conferring ease of spreading, and/or a soft, powdery, non-greasy feel, and/or for preventing pilling.

A third subject of the invention relates to a method for preparing said cosmetic compositions containing a cyclodextrin according to the invention.

### DETAILED DESCRIPTION OF THE INVENTION

Cyclodextrin

The composition according to the invention employs at least one cyclodextrin which is at least partially present in the form of solid particles.

In the present application, the term “cyclodextrin” denotes and includes any one of the otherwise known cyclodextrins, such as native and unsubstituted cyclodextrins containing from 6 to 12 glucose units bonded by covalent bonds between carbons 1 and 4, and especially alpha-, beta- and gamma-cyclodextrins containing, respectively, 6, 7 and 8 glucose units.

This term also covers “cyclodextrin derivatives,” namely molecules in which at least a portion of the hydroxyl OH groups has been converted into OR groups, where R generally denotes an alkyl group. From this perspective, cyclodextrin derivatives include especially methylated or ethylated cyclodextrins, but also those substituted with a hydroxyalkyl group, such as hydroxypropylated and hydroxyethylated cyclodextrins.

Nonetheless, due to their entirely natural character, the cyclodextrins which are preferred according to the present invention are native alpha-, beta- and gamma-cyclodextrins, i.e. which have not been modified.

According to a preferred embodiment, the cyclodextrin used in the composition according to the invention is a beta-cyclodextrin, preferably “native.”

The cyclodextrin may especially be in the form of a crystalline, quasi-crystalline or amorphous powder.

The cyclodextrin may be in a form referred to as “free,” i.e. a form in which its cavity is empty, or in the known inclusion complex form. The cyclodextrin is preferentially in a free form.

In the cosmetic compositions according to the invention, the cyclodextrin is at least partially present in the form of solid particles. By “at least partially” the applicant means preferably that at least 5% by weight of cyclodextrin, relative to the total weight of cyclodextrin in the composition, is present in the form of solid particles. Preferentially, at least 10% by weight of cyclodextrin, relatively to the total weight of cyclodextrin in the composition, is present in the form of solid particles, more preferentially at least 15% by weight, even more preferentially at least 25% by weight, yet more preferentially at least 50% by weight, even more preferentially at least 75% by weight, and most preferentially at least 95% by weight.

According to one embodiment, the content of cyclodextrin in the form of solid particles in the cosmetic or dermatological composition ranges from 0.1% to 100% by weight relative to the total weight of the cosmetic composition. Preferentially, this content ranges from 0.1 to 50% by weight, preferably from 0.1% to 25% by weight, more preferentially from 0.1% to 10% by weight, and even more preferentially from 0.1% to 5% by weight.

Aside from its presence in solid form, the cyclodextrin may be present in the composition according to the invention in dissolved form, due to the solid-liquid equilibrium, in the free state or in the inclusion complex state, but the dissolved cyclodextrin does not afford the compositions the improved sensory properties obtained with the solid cyclodextrin particles described previously.

As is known, or easily accessible using simple measurements, the solubility of a cyclodextrin in a physiologically acceptable medium, whether it comprises an aqueous and/or oily phase, is the value of the concentration of dissolved cyclodextrin at which the physiologically acceptable medium is saturated with cyclodextrin. When the cyclodextrin concentration exceeds this solubility, in other words when the physiologically acceptable medium is “saturated” with cyclodextrin, the cyclodextrin no longer dissolves in the medium, and solid cyclodextrin particles disperse, retaining their bulk solid state.

Thus, in order to guarantee a minimum content of cyclodextrin in the form of solid particles, it is possible to consider the solubility of said cyclodextrin, preferably at 20° C., in the cosmetic composition. Thus, according to a preferred embodiment, the content of cyclodextrin in the composition according to the invention is greater than the solubility of said cyclodextrin in the physiologically acceptable medium of the cosmetic composition. In particular, the content of cyclodextrin in the composition may be 5% greater than the solubility thereof in the physiologically acceptable medium of said composition, preferably 10% greater than the solubility thereof in said medium, more preferentially 15% greater than the solubility thereof in said medium, most preferentially 25% greater than the solubility thereof in said medium, and more preferentially 50% greater than the solubility thereof in said medium.

In particular, the total content of cyclodextrin present in the cosmetic or dermatological composition according to the invention represents from 1 to 20% by weight relative to the total weight of the physiologically acceptable medium of said composition, preferably from 5 to 15% by weight. In the particular case in which the cyclodextrin is a beta-cyclodextrin, and in which the physiologically acceptable medium consists of water, the cyclodextrin is preferably native, and the total content of cyclodextrin represents more than 1.8% by weight relative to the total weight of water present in the composition, which value corresponds approximately to the solubility of beta-cyclodextrin in water at 20° C.

When the cosmetic composition is a pulverulent composition, referred to as “dry” in the sense that it is in the form of a powder or a mixture of powders, the solubility of the cyclodextrin can be considered to be equal to zero. Indeed, pulverulent cosmetic compositions generally consist of a mixture of solid ingredients in the form of powders, which may contain a residual moisture content ranging from 0.01% to 20%, for example at most 16% for beta-cyclodextrin, and optionally a non-aqueous binder. This moisture is water bound to the powders, and is not available for dissolution at all, and therefore especially does not make it possible to dissolve the cyclodextrin. Thus, in the case of these dry cosmetic compositions, the cyclodextrin is always present in an amount greater than the solubility thereof in said compositions.

In the context of the present invention, the cyclodextrin is at least partially present in the cosmetic or dermatological composition in the form of solid particles of volume average size d(4.3), measured by laser particle size analysis, of less than or equal to 20 μm, more preferentially less than or equal to 12 μm, and most preferentially less than or equal to 8 μm.

According to a preferential embodiment, the volume average size of the solid-cyclodextrin particles is between 2 μm and 20 μm, preferentially between 3 μm and 12 μm, and most preferentially between 4 μm and 8 μm.

The solid cyclodextrin particles are characterized by a size distribution by volume, measured by laser diffraction particle size analysis, of which the characteristic diameters d(10), d(50) and d(90) are such that:
the diameter d(10) is less than or equal to 5.0 μm, preferentially less than or equal to 2.5 μm,
the diameter d(50) is less than or equal to 15.0 μm, preferentially less than or equal to 10.0 μm, and
the diameter d(90) is less than or equal to 30.0 μm, preferentially less than or equal to 25.0 μm.

The characteristic diameters d(10), d(50) and d(90), as defined in standard ISO 13320:2009 with the notations x10, x50 and x90, are the diameters of particles corresponding, respectively, to 10%, 50% and 90% of the cumulative size distribution by volume.

Even more preferentially, the solid-cyclodextrin particles are characterized by a size distribution by volume, measured by laser diffraction particle size analysis, having a coefficient of variation of less than or equal to 100%, preferentially less than or equal to 90%, and most preferentially less than or equal to 73%. As defined in standard ISO 13320:2009, the coefficient of variation of the particle size distribution is the standard deviation of the particle size distribution divided by the volume average size d(4.3), also referred to as the volume average diameter.

The solid cyclodextrin particles may be of any even or uneven geometric shape, and may be highly individualized crystals of cyclodextrin or agglomerates of crystals of cyclodextrin bound to one another by crystalline bridges.

The volume average size, generally denoted d4.3 or d(4.3), is determined based on size distributions by volume measured by laser diffraction particle size analysis, for example using a laser particle size analyzer from the MasterSizer® range, for example “Mastersizer 2000™,” “Mastersizer 3000™,” “Mastersizer 3000E™,” from Malvern Instruments®, or a “Particula LA960” laser particle size analyzer from Horiba©. These methods of measuring by laser diffraction may be implemented via the wet route or the dry route. When the wet route is used, it is recommended to use 2-propanol as measuring fluid.
According to this preferential embodiment, the cyclodextrin is therefore present in the form of solid particles, the size distribution by volume of which, measured by laser diffraction particle size analysis, has the following characteristics:
a. A volume average size d(4.3) of less than or equal to 20 μm, preferentially less than or equal to 12 μm, and most preferentially less than or equal to 8 μm,
b. A coefficient of variation of less than or equal to 100%, preferentially less than or equal to 90%, and most preferentially less than or equal to 73%.

Fields of Cosmetics to Which the Invention Relates

The cosmetic compositions according to the invention are able to be used in all fields of cosmetics, which cover skin care and protection, bodily hygiene, hair treatments, oral-dental treatments, perfumes, eaux de toilette and eaux de cologne, hair-removing treatments, and make-up and make-up removal for eyelids, eyelashes, cheeks, lips, nails or other parts of the body.

Preferentially, the cosmetic compositions according to the invention are used in skin care and protection, hair care, oral-dental treatments, make-up and make-up removal, and hair-removing treatments.

In the field of skin or lip care, the cosmetic compositions according to the invention may be creams, emulsions, lotions, gels and oils for the skin, anti-aging or anti-wrinkle products, beauty masks, solar care products, self-tanning products, skin-whitening products or else lip balms.

In the field of bodily hygiene and body care, the cosmetic compositions according to the invention may be soaps, liquid soaps, deodorant soaps, shower gels, shampoos, bath powders, preparations for bath and shower in the form of salts, mousses, oils, dental hygiene products such as toothpastes and mouthwashes, products for shaving, and make-up removal products.

In the field of hair, the cosmetic compositions may be products for adding waves or smoothing the hair or hairstyling products, hair setting products, products for cleaning the hair such as lotions or powders or shampoos, hair upkeep products such as lotions or creams or oils, hairstyling products in the form of lotions or hairsprays or waxes, or hair dyeing products.

In the field of make-up, the cosmetic compositions according to the invention may be foundations in liquid, paste or powder form, and also make-up products intended to be applied to the eyelashes, nails, cheeks or lips, such as lipsticks.

Sensory Powder

In the cosmetic composition according to the invention, the cyclodextrin acts as a sensory powder. By “sensory powder” the applicant means to designate a cosmetic ingredient present in solid form, preferentially in the form of a powder, having characteristics of form, surface state, structure or mechanical properties, especially flexibility and hardness, and of particle size, affording it beneficial sensory effects. In the context of the present invention, these beneficial sensory effects may be a reduction in the greasy sensation, a drier and softer feel, and a non-tacky finish.

Forms of Administration

The cosmetic compositions according to the invention may be present in the form of a gel, such as an aqueous or aqueous-alcoholic gel, of an aqueous and/or oily liquid dispersion, such as an emulsion, a suspension, a lotion, a serum, an aerosol or a mousse, or else in the form of a paste or a powder, or in the form of a solid.

According to one embodiment of the invention, the cosmetic composition is a water-in oil emulsion or an oil-in-water emulsion. While water-in-oil emulsions provide a greasy, slippery and non-soft feel due to their ability to form a lipid film on the surface of the skin, oil-in-water emulsions generally provide a softer and less greasy feel than said water-in-oil emulsions. The emulsions may be present in different usage forms depending on their formulation, from fluid to thick: serum, milk, mousse, fluid or thick cream, or gel. Such emulsions have several fields of application: face and body milks, solar care products (sprayable emulsions and milks), make-up (mascaras, foundations, eyeshadows, cream), facial care creams, cold creams, hand and body creams, haircare (conditioners, shampoos, creams and cream masks).

The emulsion according to the invention is preferentially an oil-in-water emulsion in which the total content of cyclodextrin is between 1.8% and 10% by weight relative to the total weight of water. Preferentially, in this embodiment said cyclodextrin is a beta-cyclodextrin. According to a preferred embodiment, the emulsion may be an emulsion stabilized by an emulsifying system consisting of beta-cyclodextrin according to the invention and an emulsifier of natural origin as defined below.

According to another embodiment, the cosmetic composition according to the invention is a liquid, aqueous or fatty dispersion, for example a lotion, which contains a liquid, aqueous or fatty phase, and a pulverulent phase, said pulverulent phase comprising at least one cyclodextrin which is at least partially present in the form of solid particles according to the invention.

According to another embodiment, the cosmetic composition according to the invention is a paste for cosmetic use, present as a semi-solid medium consisting of a highly viscous continuous phase in which solid cyclodextrin particles are dispersed. It may for example take the form of a toothpaste or a lip balm.

According to another embodiment, the cosmetic composition according to the invention is a solid, such as a lipstick, a deodorant stick, or soap.

According to another embodiment, the cosmetic composition according to the invention is a powder for cosmetic use, which comprises a pulverulent phase comprising pigments and fillers, and a fatty phase comprising fatty substances which act as binders to confer defined properties of density, emollience and softness during application of the finished product. Such a pulverulent composition is in the form of eyeshadow, blusher, anti-wrinkle product, face and body powder, and products for making up the face and body.

In this case of cosmetic composition in powder form, the composition may be obtained by a “slurry” type method i.e. by dispersing a pulverulent phase in an aqueous-alcoholic solvent phase, then evaporating the solvent phase.

Thus, according to a preferred embodiment of the invention, the cosmetic or dermatological composition is present in the form of a skincare product, a hair care product, an oral-dental product, a make-up or a hair-removing treatment.

Components of the Cosmetic Composition

Physiologically Acceptable Medium

The composition according to the invention also comprises a physiologically acceptable, and preferably cosmetically acceptable, medium, i.e. one which has no harmful side effects and in particular which does not produce redness, heat, pain or tingling which are unacceptable for a user of cosmetic products.

The physiologically acceptable medium comprises for example a fatty phase and/or an aqueous phase.

Fatty Phase

The fatty phase may contain at least one oil.

For the purposes of the present invention, “oil” is understood to mean a compound which is liquid at room temperature (25° C.) and which, when it is introduced at an amount of at least 1% by weight into the water at 25° C., is not at all soluble in the water, or soluble to an extent of less than 10% by weight, relative to the weight of oil introduced into the water.

The liquid fatty phase advantageously comprises one or more nonvolatile oils which afford an emollient effect on the skin. Mention may be made of fatty esters such as cetearyl isononoate, isotridecyl isononoate, isostearyl isostearate, isopropyl isostearate, isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate or lactate, 2-diethylhexyl succinate, diisostearyl malate, triacetin, tricaprin, caprylic/capric acid triglycerides, coco caprate and caprylate mixture, benzoates of C12 to C15 alcohols, glycol esters such as butylene glycol cocoate, glyceryl triisostearate, tocopherol acetate, higher fatty acids such as myristic acid, palmitic acid, stearic acid, behenic acid, oleic acid, linoleic acid, linolenic acid or isostearic acid, higher fatty alcohols such as oleic alcohol, vegetable oils such as avocado oil, camellia oil, hazelnut oil, tsubaki oil, cashew nut oil, argan oil, soybean oil, grape seed oil, sesame oil, corn oil, wheatgerm oil, rapeseed oil, sunflower oil, cottonseed oil, jojoba oil, peanut oil, olive oil and mixtures thereof, vegetable butters such as shea butter, camellia butter.

According to a preferred embodiment, the composition according to the invention comprises a nonvolatile oil selected from the coco caprate and caprylate mixture, glycol esters such as butylene glycol cocoate and the mixture thereof.

These oils may be oils of hydrocarbon-based or silicone type, such as paraffin oils, squalane, petroleum jelly, dimethyl siloxanes and mixtures thereof.

The liquid fatty phase may also optionally comprise volatile oils. Volatile oil is intended to mean an oil which is capable of evaporating from the skin in less than one hour at room temperature and atmospheric pressure. The volatile oils may for example be selected from silicone oils or short-chain fatty acid triglycerides in order to reduce the greasy feel.

The composition according to the invention preferably only contains oils of renewable origin and especially oils of vegetable origin, preferably refined.

The amount of oil contained in the composition according to the invention preferably represents from 10 to 80% of the total weight of this composition.

The fatty phase of the composition according to the invention may further advantageously contain at least one fatty phase structuring agent such as a lipophilic gelling agent.

The fatty phase may also contain one or more waxes and/or one or more pasty compounds.

“Pasty compound” is understood to mean lipophilic fatty substances which, like waxes, are capable of undergoing a reversible liquid-solid state change and which have, in the solid state, anisotropic crystalline organization, but which differ from waxes in that they contain, at a temperature of 23° C., a liquid fraction and a solid fraction.

“Wax” is intended to mean a fatty substance having reversible liquid-solid state change and having a melting point of greater than 30° C. and generally less than 90° C., which is liquid under the preparation conditions of the composition and which has anisotropic crystalline organization in the solid state. The waxes used according to the invention may consist of polar or apolar waxes or a mixture of these two. “Apolar” is intended to mean a wax containing only carbon, hydrogen and/or phosphorus atoms, and in particular a hydrocarbon.

The polar waxes may be selected from animal waxes, vegetable waxes, and synthetic or silicone waxes containing polar groups such as esters. Mention may thus be made of carnauba wax, candellila wax, beeswax (Cera alba), Chinese insect wax (Ericerus pela), Japan wax, sumac wax, montan wax, triesters of C8-C20 acids and glycerin, such as glyceryl tribehenate, acetylated glycol stearate, sold especially by VEVY under the trade name CETACENE, and mixtures thereof. These waxes may especially be used in the form predispersed in an oil, as is the case for the mixture of candelilla wax and jojoba seed oil sold by INA TRADING under the trade name GREEN GREASE.

The fatty phase of the composition according to the invention may also comprise at least one film-forming polymer, capable of providing hold and/or non-transfer properties and/or shine to the make-up provided by the composition.

Aqueous Phase

The composition according to the invention may further comprise an aqueous phase comprising water and optionally one or more water-miscible organic solvents and/or one or more rheology agents.

The composition according to the invention may especially comprise a water content ranging from 10 to 50% by weight relative to the total weight of the composition.

The rheology agent may especially be an agent for thickening the aqueous phase, a gelling agent or a suspending agent, for example plant-derived gums such as gum arabic, konjac gum, guar gum or derivatives thereof; gums extracted from algae such as alginates or carrageenans; gums resulting from microbial fermentation such as xanthans, mannans, scleroglucans or derivatives thereof, cellulose and derivatives thereof such as carboxymethylcellulose or hydroxyethylcellulose; starch and derivatives thereof such as modified starches, especially acetylated, carboxymethylated, octenylsuccinates or hydroxypropylated; synthetic polymers such as polyacrylic acids or carbomers.

According to a preferred embodiment, the rheology agent is xanthan gum.

The composition according to the invention preferably comprises a rheology agent selected from natural polysaccharides derived from plants or from fermentation, optionally modified.

Surfactants

The composition according to the invention may also comprise one or more surfactants, preferably selected from water-in-oil (W/O) or oil-in-water (O/W) emulsifiers, preferably of natural origin.

The oil-in-water emulsifier may especially be selected from optionally polyethoxylated sorbitan esters, glycerol fatty acid esters, sucrose fatty acid esters or polyesters, polyethylene glycol fatty acid esters, polyether-modified polysiloxanes, polyethylene glycol fatty alcohol ethers, alkyl polyglycosides and hydrogenated lecithin, this list being non-limiting.

The water-in-oil (W/O) emulsifier may be selected from non-ethoxylated polyol fatty esters, and especially from non-ethoxylated fatty esters of glycerol, of polyglycerols, of sorbitol, of sorbitan, of anhydrohexitols, such as in particular isosorbide, mannitol, xylitol, erythritol, maltitol, sucrose, glucose, polydextrose, hydrogenated glucose syrups, dextrins and hydrolyzed starches.

The W/O emulsifier may be selected from non-ethoxylated polyol fatty esters obtained from fatty acids or by transesterification from oil or oil mixtures. The fatty acids used comprise from 8 to 22 carbon atoms, preferably from 10 to 18 carbon atoms, and in particular from 12 to 18 carbon atoms. These acids may be linear or branched, saturated or unsaturated and have one or more lateral hydroxyl functions. The oils may be saturated or unsaturated, from liquid to solid at room temperature, and optionally have hydroxyl functions, preferably an iodine value of between 1 and 145 and in particular from 5 to 105.

The W/O emulsion of natural origin may also be selected from products which biodegrade naturally in a natural hydrated medium, especially with a hydrophilic-lipophilic balance (HLB) of between 1.5 and 6, preferably between 2 and 5 and better still between 3 and 5.

The W/O emulsifier of natural origin may in particular be selected from glycerol fatty esters, and especially from glyceryl oleates, stearates, and isostearates, for example the following products: glyceryl laurate HLB 5.2, glyceryl oleate HLB 4, such as IMWITOR 948, glyceryl isostearate Schercemol GMIS HLB 3.5 from Lubrizol Schercemol and glyceryl monostearate HLB 3.5 from Sympatens-GMS.

It may also be selected from sorbitan or sorbitol fatty esters, especially from sorbitan laurates, palmitate, oleates, stearate, isostearates, for example the products sorbitan trioleate Kosteran-O/3 HLB 1.8; sorbitan oleate MONTANE 80 VG or SPAN 80-LQ-(RB) or Kosteran-O/1 HLB 4.3; sorbitan isostearate Kosteran-I/1 HLB 4.3, sorbitan stearate Kosteran-S/1 HLB 4.7; sorbitan monopalmitate HLB 6,6; sorbitan laurate Kosteran-L/1 HLB 8.6.

It may also be selected from sucrose fatty esters, for example sucrose distearate SP60-C from Sisterna, sucrose polystearate SP10-C from Sisterna, sucrose cocoate HLB 6.

The W/O emulsifier of natural origin may in particular be selected from polyglycerol esters, and preferably from esters resulting from the reaction of polyglycerols comprising from 2 to 12 glycerol units, preferably from 3 to 6 glycerol units with at least one partially hydrogenation or non-hydrogenated vegetable oil with an iodine value of between 1 and 1 5, and in particular from 5 to 10. This may in particular be oleic, stearic, isostearic and ricinoleic polyglycerol esters, and in particular the following products: polyglyceryl-4 isostearate HLB 3 (such as HYDRIOL® PGI from HYDRIOR), polyglyceryl-10 pentaoleate HLB 3.5 (such as DECAGLYN 5-OV), polyglyceryl-6 polyricinoleate (such as HEXAGLYN PR-15), polyglyceryl-2 sesquiisostearate HLB˜4 (such as Hostcerin DGI from Clariant and Dermofeel® GO soft from Evonik Dr. Straetmans), polyglyceryl-3 ricinoleate HLB 3.5, polyglyceryl-3 polyricinoleate HLB 4 (such as IMWITOR 600), polyglyceryl-3 polyricinoleate HLB: ˜4 (such as Dermofeel® PGPR from Evonik Dr. Straetmans GmbH) polyglyceryl-2 sesquioleate HLB 4 (such as Dermofeel® GO soft from Evonik Dr. Straetmans GmbH), polyglyceryl-2 diisostearate (such as Emulpharma® PG20 from Res Pharma), polyglyceryl-3 diisostearate HLB: 5.5 (such as Plurol® Diisostearique CG from Gattefossé, Lameform® TGI from BASF, IMWITOR® PG3 DIS from 101 Oleo GmbH, Cithrol™ PG32IS from Croda, DUB ISO G3 from Stéarinerie Dubois, Polyaldo® 3-1-S from Lonza, Jolee 7245 from Oléon and MASSOCARE PG3D from Masso), polyglyceryl-3 oleate HLB 6.2 (such as I-MUL PGO 31 from Ivanhoe Industries), polyglyceryl-3 monostearate LB 7.2, polyglyceryl-4 oleate HLB 8 (such as HYDRIOL® PGMO.4 from HYDRIOR), polyglyceryl-5 dioleate HLB 8 (such as Dermofeel® G 5 DO from Evonik Dr. Straetmans GmbH), polyglyceryl-2 dipolyhydroxystearate (such as Dehymuls PGPH from Cognis), polyglyceryl-2 diisostearate (such as Emulpharma PG20 from Res Pharma), polyglyceryl-3 cocoate (such as Emulpharma® Ecotech from Res Pharma).

According to a preferred embodiment, the composition according to the invention comprises, as W/O emulsifier, the ester resulting from the reaction of polyglycerol-3 and isostearic acid (INCI name: polyglyceryl-3 diisostearate).

The W/O emulsifier of natural origin may consist of mixtures of fatty esters, especially polyglycerol fatty esters, sorbitan fatty esters or glucose esters such as, in particular, mixtures such as the products Nikkomulse WO-NS from Nikko Chemicals (Polyglyceryl-6 polyricinoleate, polyglyceryl-2 isostearate, disteardimonium hectorite), Tego Care LTP from Evonik (sorbitan laurate, polyglyceryl-4 laurate, dilauryl citrate), Sympatens-W/4500 (sorbitan oleate, polyglyceryl-3 polyricinoleate), Sympatens-O/2500 G (sorbitan stearate, methylglucose sesquistearate), Symbio®muls WO from Dr. Straetmans (polyglyceryl-3 polyricinoleate, sorbitan sesquioleate, cetyl ricinoleate, glyceryl caprate, cera alba, magnesium stearate, aluminum stearate), Ecomuls 2 in 1 from Natura-Tec (glyceryl oleate, polyglyceryl-3-polyricinoleate, olea europaea (olive) oil unsaponifiables), HIPEgel Olea from Alchemy (glycerin, isopropyl palmitate, water, sucrose stearate, sucrose laurate).

According to a preferred embodiment, the emulsifiers used in the compositions of the invention are of natural origin. The term “natural origin” denotes any molecule originating from renewable resources, especially extracted from or secreted by plants, microorganisms or algae and able, after physical, chemical or enzymatic modification, to be used in a cosmetic composition.

The surfactants implemented in the composition according to the invention are present at an amount ranging from 0.01 to 5% by weight relative to the total weight of the composition, preferably from 0.1 to 1% by weight.

Dyestuff

The composition according to the invention may further comprise at least one dyestuff selected from water-soluble or liposoluble dyes, fillers with the effect of coloring and/or opacifying the composition and/or coloring the lips, such as pigments, nacres, lakes (water-soluble dyes adsorbed on an inert mineral support) and mixtures thereof. These dyestuffs may be optionally surface-treated by a hydrophobic agent such as silanes, silicones, fatty acid soaps, C9-15 fluoroalcohol phosphates, acrylate/dimethicone copolymers, C9-15 fluoroalcohol phosphate/silicon mixed copolymers, lecithins, carnauba wax, polyethylene, chitosan and optionally amino acids which could be acylated such as lauroyl lysine, disodium stearoyl glutamate and aluminum acyl glutamate. The pigments may be mineral or organic, natural or synthetic. Examples of pigments are especially iron, titanium or zinc oxides, and also composite pigments and goniochromatic, pearlescent, interference, photochromic or thermochromic pigments, with this list being non-limiting.

Advantageously, when it contains one or more pigments, the composition according to the invention further contains at least one dispersant such as butylene glycol cocoate or diisostearyl malate.

Fillers

The composition used according to the invention may further contain at least one filler other than the cyclodextrin previously described. This term is intended to mean particles of any form (especially spherical or lamellar), mineral or organic, which are insoluble in the composition. Examples of fillers are talc, mica, silica, kaolin, boron nitride, starch, starch modified with octenylsuccinic anhydride, polyamides, silicone resins, silicone elastomer powders and acrylic polymer powders, in particular powders of poly(methyl methacrylate) or powders of styrene acrylate copolymer (Sunsphere Powders from Dow). The fillers may especially consist of several layers of different chemical nature and/or physical form, and especially be present in the form of lamellae coated in spherical fillers. They may be modified using different surface treatments. An example of surface-treated filler consists of silica modified by an ethylene/methacrylate copolymer, sold especially by KOBO under the trade names DSPCS 20N-I2, 3H-I2 and I2.

Active Ingredients

The composition used according to the invention may further contain one or more hydrophilic or lipophilic active ingredients.

Customary Additional Cosmetic Ingredients

The composition according to the invention may also comprise any customary cosmetic ingredient which may be especially be selected from antioxidants, fragrances, preservatives, neutralizers, surfactants, sunscreens, vitamins, moisturizers, self-tanning compounds, anti-wrinkle active ingredients, emollients, hydrophilic or lipophilic active ingredients, anti-free-radical agents, deodorizing agents, sequestrants, film-forming agents, and mixtures thereof.

In particular, the composition according to the invention may comprise at least one sunscreen.

The Uses of Cyclodextrin According to the Invention

According to a first embodiment, one of the subjects of the present invention relates to the use, in a cosmetic or dermatological composition, of at least one cyclodextrin which is at least partially present in the form of solid particles dispersed in a physiologically acceptable medium, characterized in that the cyclodextrin particles have a volume average size d(4.3), measured by laser particle size analysis, of less than or equal to 20 μm, more preferentially less than or equal to 12 μm, and most preferentially less than or equal to 8, and a size distribution by volume, measured by laser diffraction particle size analysis, of which the characteristic diameters d(10), d(50) and d(90) are such that:
a. the diameter d(10) is less than or equal to 5.0 μm, preferentially less than or equal to 2.5 μm,
b. the diameter d(50) is less than or equal to 15.0 μm, preferentially less than or equal to 10.0 μm, and
c. and the diameter d(90) is less than or equal to 30.0 μm, preferentially less than or equal to 25.0 μm,
for improving the sensory properties of said composition, preferentially for conferring ease of spreading, and/or a soft, powdery, non-greasy feel, and/or for preventing pilling.

Moreover, this use enables a clear reduction in the squeaky sensation upon application, and also a reduction in, or even a disappearance of, pilling, depending on the skin type.

In particular, the cyclodextrin used in this use is as described above.

Method for Preparing a Cosmetic Composition According to the Invention

A final subject of the present invention consists of a method for preparing a cosmetic composition, preferably an oil-in-water emulsion, comprising the following steps:
a) providing a physiologically acceptable medium,
b) dispersing, in said physiologically acceptable medium, at least one cyclodextrin having a volume average size d(4.3), measured by laser particle size analysis, of less than or equal to 20 μm, more preferentially less than or equal to 12 μm, and most preferentially less than or equal to 8, and a size distribution by volume, measured by laser diffraction particle size analysis, of which the characteristic diameters d(10), d(50) and d(90) are such that:
a. the diameter d(10) is less than or equal to 5.0 μm, preferentially less than or equal to 2.5 μm,
b. the diameter d(50) is less than or equal to 15.0 μm, preferentially less than or equal to 10.0 μm, and
c. the diameter d(90) is less than or equal to 30.0 μm, preferentially less than or equal to 25.0 μm.

According to one variant, the method according to the invention is the content of cyclodextrin in the physiologically acceptable medium is 5% greater than the solubility thereof in said medium, preferably 10% greater than the solubility thereof, more preferentially 25% greater than the solubility thereof, and more preferentially 50% greater than the solubility thereof.

### EXAMPLES

### Example 1

### Emulsion

An O/W emulsion produced according to the invention was compared with a “reference” emulsion, not produced in accordance with the invention, having the following composition:

TABLE 1
Composition of the Emulsion

| | % by |
|---|---|
| Component/INCI name | weight |
| Sunflower oil/Helianthus annuus seed oil | 30 |
| Beta-cyclodextrin/Cyclodextrin | 2.5 |
| Sorbitol (powder) | 1 |
| Polyglyceryl-3 diisostearate | 0.5 |
| Demineralized water/Aqua | 64.3 |
| Xanthan gum/Xanthan gum | 0.7 |
| Preservative | 1 |

The reference emulsion is obtained using a beta-cyclodextrin sold under the name “Beauté by Roquette® CD102” by the applicant. The emulsion according to the invention is obtained using this same beta-cyclodextrin, dry milled beforehand to have a volume average size d(4.3) of less than 20 μm: the beta-cyclodextrin thus obtained is termed “ultra-fine.” The particle size characteristics of these two beta-cyclodextrins are presented in table 2.

TABLE 2
Particle Size Characteristics of the Beta-Cyclodextrins

| | | Beta-cyclodextrin |
|---|---|---|
| | Beta-cyclodextrin | according to the |
| | Beauté by Roquette ® | invention, referred to |
| | CD102 (reference) | as “ultra-fine” |
| d(4.3) (μm) | 94.83 | 11.43 |
| d10 (μm) | 21.81 | 2.378 |
| d50 (μm) | 85.81 | 9.716 |
| d90 (μm) | 182.2 | 22.85 |
| Coefficient of | 64.7% | 72.3% |
| variation (%) | | |

The protocol for preparing the emulsions is as follows. A thickener, in this instance xanthan gum, is dispersed in water at 40° C. with stirring with a deflocculating blade at 500 rpm. Separately, the beta-cyclodextrin is mixed with the sorbitol and the polyglyceryl-3 diisostearate. This mixture is then added into the water containing the thickener, with stirring at 1000 rpm, in order to obtain an aqueous phase.

The amount of beta-cyclodextrin is fixed at 2.5% by weight of the composition for the two emulsions.

Sunflower oil constitutes the oily phase; it is heated to 40° C.

The oily phase is then emulsified in the aqueous phase at 40° C. with stirring at 3000 rpm for 20 minutes.

This is left to cool, and at room temperature (20° C.), a phenoxyethanol-based preservative is finally added.

For each of the emulsions, the physicochemical characteristics are measured, namely viscosity and droplet size, and the sensory characteristics are measured, namely opacity, whiteness, shine, fluidity, smooth-flowingness, glide, whitening, spreading, greasiness, tackiness, softness, squeakiness, penetrability, powderiness, and pilling.

The viscosity is measured using a Brookfield DV-II+Pro viscometer. A spindle of fixed size (spindles SP2 and SP7 used depending on the viscosity levels, in accordance with the appliance recommendations) is rotated at a speed of 20 rpm in contact with the sample produced. The resistance of the product to this rotational movement is recorded for one minute and converted into millipascal-seconds. For each sample, the viscosity is measured three times and the arithmetic mean of the three values is retained.

The droplet size is observed with an optical microscope at 10× magnification. The microscope used is a LEICA DMLS.

The sensory characteristics are evaluated by a panel of ten people who are experts in analyzing the texture of cosmetic products.

The three first descriptors evaluated are the visual characteristics of the product. The examination is carried out in the pot, under a lamp fitted with a 5500 K-temperature bulb:

The descriptor fluid is evaluated when pressure is applied to the product between the thumb and the index finger. If there is no resistance, and it flows off the fingers and moves away from the pressure zone, it is fluid; if the opposite is true, it is non-fluid.

When the product is spread, four other descriptors are evaluated. The product is examined, after having deposited 50 to 100 μl of the studied product on the hand, under the lamp during the spreading thereof in 10 rotations.

The descriptor powderiness is evaluated between the 2^{nd} and the 5^{th} rotation. The fingers slide well over the skin. The product is perceived as a powdery substance on the skin.

The spread is evaluated, after having deposited 50 to 100 μl of the product on the hand, by examining the product during the spreading thereof in 10 rotations, under a lamp. The spread is greater the less resistance to movement there is between the 5th and 10th turn on the hand.

The final descriptors are evaluated after having carried out the 10 rotations.

For the four following descriptors, the examination is carried out under the lamp, on the skin, 1 minute after spreading 50 to 100 μl of the product.

The softness descriptor is evaluated by sliding over the skin; a dry and slippery feel is perceived.

The squeakiness descriptor is evaluated by sliding the thumb over the index finger; resistance is perceived and a squeaky sound is heard.

For the two following descriptors, the examination is carried out under the lamp, on the skin, 2 minutes after spreading 50 to 100 μl of the product.

The penetrability descriptor of the product is evaluated by sliding over the skin. A panel of evaluators then evaluates the amount of product residue recovered.

Pilling is evaluated by performing a mechanical rubbing action on the skin; the product leads to pilling.

TABLE 3
Improves sensory perceptions compared to the reference emulsion

| | Emulsion according to the invention, |
|---|---|
| | with ultra-fine beta-cyclodextrin, |
| Sensory | compared to the emulsion |
| Perception | with Beauté by Roquette ® CD102 |
| Fluidity | Less fluid |
| Powderiness | More powdery |
| Spread | Easier |
| Softness | Softer feel |
| Squeakiness | Markedly less squeaky |
| Penetrability | Quicker penetration |
| Pilling | Fewer, or no, pills |

The criteria of sensory properties which are improved in the emulsion according to the invention are: fluidity, powderiness, spread, greasiness, softness, squeakiness, penetrability, pilling.

### Example 2

### Sun Cream

The sensory profile of a sun cream formulation prepared with a beta-cyclodextrin of mean diameter of 200 μm (Beauté by Roquette® CD102 of example 1) was compared with one prepared with a beta-cyclodextrin of volume average size of 11.43 μm (ultra-fine beta-cyclodextrin of example 1) according to the following procedure.

First of all, the phase Al is prepared according to the composition in table 4: the Sunsphere powder is dispersed in water and Cetiol C5, then heating at 55° C. is carried out, with stirring at 2000 rpm with a rotor-stator for 15 minutes.

TABLE 4
composition of phase A1

| | | | | wt % |
|---|---|---|---|---|
| | | | | relative |
| Phase | Trade name | Supplier | INCI name | to total |
| A1 | Demineralized | Cooper | Aqua | 36.44 |
| | water | | | |
| | EDETA BD | AMI Chimie | Disodium EDTA | 0.10 |
| | Sunsphere | Dow | Styrene/acrylate | 5.00 |
| | powder | (Univar) | copolymer | |
| | Cetiol C5 | AMI Chimie | Coco-caprylate | 1.00 |

Separately, phase A2 was prepared by weighing all the ingredients in a dish, then it was added to the phase Al with rotor-stator stirring at 2000 rpm for 15 min. The phase A1+A2 is kept at 70° C. with stirring.

TABLE 5
composition of phase A2

| | | | | wt % |
|---|---|---|---|---|
| | | | | relative |
| Phase | Trade name | Supplier | INCI name | to total |
| A2 | Sepinov | Seppic | Hydroxymethyl | 0.20 |
| | WEO | | Acrylate/Sodium | |
| | | | Acryloyldimethyl | |
| | | | Taurate Copolymer | |
| | Microcare | Thor | Glycerin & | 1.00 |
| | PHC | | Chlorphenesin & | |
| | | | Phenoxyethanol | |
| | Xanthan gum | Interchimie | Xanthan gum | 0.30 |
| | Glycerin | Cooper | Glycerin | 3.00 |
| | codex | | | |
| | Romol AFSK | Saci cfpa | Potassium cetyl | 0.30 |
| | | | phosphate | |

Next, phase B1 is prepared by heating the composition of table 6 at 70° C. with magnetic stirring for 10 minutes.

TABLE 6
composition of phase B1

| | | | | wt % |
|---|---|---|---|---|
| | | | | relative |
| Phase | Trade name | Supplier | INCI name | to total |
| B1 | Cetiol C5 | AMI Chimie | Coco-caprylate | 2.00 |
| | Cocoate BG | Gattefossé | Butylene glycol cocoate | 2.00 |
| | Parsol 1789 | IES | Butyl | 5.00 |
| | | Ingrédients | Methoxydibenzoylmethane | |
| | Parsol EHS | IES | Ethylhexyl Salicylate | 5.00 |
| | | Ingrédients | | |
| | Parsol MCX | IES | Ethylhexyl | 7.50 |
| | | Ingrédients | Methoxycinnamate | |
| | Parsol HMS | IES | Homosalate | 9.00 |
| | | Ingrédients | | |
| | Solastay S1 | Azelis | Ethylhexyl methoxycrylene | 3.00 |
| | Hallbrite | Azelis | Butyl Octyl Salicylate | 4.00 |
| | BHB | | | |
| | Tinogard TL | AMI Chimie | Benzotriazolyl Dodecyl P- | 1.00 |
| | | | Cresol | |
| | Covanol | Sensient | Iron oxides (CI77491) & | 0.12 |
| | Red ON | | Octyldodecanol & Stearic | |
| | 3780 | | acid & Magnesium | |
| | | | hydroxide & Aluminum | |
| | | | hydroxide & Sorbitan | |
| | | | oleate | |
| | Covanol | Sensient | Iron oxides (CI77492) & | 0.53 |
| | Yellow ON | | Octyldodecanol & Stearic | |
| | 1782 | | acid & Magnesium | |
| | | | hydroxide & Aluminum | |
| | | | hydroxide & Sorbitan | |
| | | | oleate | |
| | Oleic | Oléon | Helianthus annuus seed | 3.00 |
| | sunflower oil | | oil | |

Keeping phase B1 at 70° C. with stirring, phase B2 is added, which was prepared separately and formed according to the composition of table 7; this is kept under stirring for 10 minutes, after which time the phase B1+B2 is emulsified in phase A.

TABLE 7
composition of phase B2

| | | | | wt % |
|---|---|---|---|---|
| | | | | relative |
| Phase | Trade name | Supplier | INCI name | to total |
| B2 | Beauté by | Roquette | Cyclodextrin | 2.5 |
| | Roquette ® | Frères | | |
| | CD102 | | | |
| | Plurol | Gattefossé | Polyglyceryl 3- | 0.5 |
| | | | diisostearate | |
| | Neosorb | Roquette | Sorbitol | 1 |
| | | Frères | | |

To emulsify phase B1+B2 in phase A, the whole of phase B1+B2 is poured into phase A at 55° C. with rotor-stator stirring at 2500 rpm, the temperature is then kept at 55° C. for 5 minutes, and for 10 minutes in a cold-water bath. The speed is reduced to 1200 rpm and stirring is continued until the emulsion is at room temperature in the cold water bath.

Phase C is then added, then coloring is carried out with phase D.

A colored and perfumed cream is then obtained. When it is applied to the skin, the cream pills; it is comfortable, but a squeaky effect is noted during the spreading thereof.

TABLE 8
composition of phases C and D

| | | | | wt % |
|---|---|---|---|---|
| | | | | relative |
| Phase | Trade name | Supplier | INCI name | to total |
| C | Plouf RL G | Robertet | Fragrance | 0.50 |
| | 113 23705 | | | |
| D | Covarine white | Sensient | CI 77891 (and) Glycerin | 4.00 |
| | WN 9787 | | (and) Xanthan Gum | |
| | | | (and) Sodium Citrate | |
| | | | (and) Aqua | |
| | Covarine black | Sensient | CI 77499 & Glycerin & | 0.01 |
| | WN 9798 | | Xanthan gum & Sodium | |
| | | | citrate & Aqua | |

A sun cream is prepared according to the preceding protocol, substituting “Beauté by Roquette® CD102” with the “ultra-fine” beta-cyclodextrin of example 1. The cream is applied to the skin: it no longer pills and the cream is more comfortable. A slight squeaky effect remains at the end of the penetration of the cream, but it is markedly less present than with the reference formulation comprising the beta-cyclodextrin “Beaute by Roquette® CD102.”

1. A cosmetic or dermatological composition comprising at least one cyclodextrin which is at least partially present in the form of solid particles dispersed in a physiologically acceptable medium, wherein the cyclodextrin particles have:
a volume average size d(4.3), measured by laser particle size analysis, of less than or equal to 20 μm, more preferentially less than or equal to 12 μm, and most preferentially less than or equal to 8 μm, and
a size distribution by volume, measured by laser diffraction particle size analysis, of which the characteristic diameters d(10), d(50) and d(90) are such that:
a. The diameter d(10) is less than or equal to 5.0 μm, preferentially less than or equal to 2.5 μm,
b. And the diameter d(50) is less than or equal to 15.0 μm, preferentially less than or equal to 10.0 μm,
c. And the diameter d(90) is less than or equal to 30.0 μm, preferentially less than or equal to 25.0 μm.

2. The cosmetic or dermatological composition according to claim 1, wherein the solid cyclodextrin particles have a size distribution by volume, measured by laser diffraction particle size analysis, having a coefficient of variation of less than or equal to 100%, preferentially less than or equal to 90%, and most preferentially less than or equal to 73%.

3. The cosmetic or dermatological composition according to claim 1, wherein at least 5% by weight of cyclodextrin, relative to the total weight of cyclodextrin present in the composition, is in the form of solid particles, preferentially at least 10% by weight, more preferentially at least 15% by weight, more preferentially at least 25% by weight, and even more preferentially at least 50% by weight.

4. The cosmetic or dermatological composition according to claim 1, wherein the content of cyclodextrin in the composition is 5% greater than the solubility thereof in the physiologically acceptable medium of said composition, preferably 10% greater than the solubility thereof in said medium, more preferentially 15% greater than the solubility thereof in said medium, most preferentially 25% greater than the solubility thereof in said medium, and more preferentially 50% greater than the solubility thereof in said medium.

5. The cosmetic or dermatological composition according to claim 1, wherein the cyclodextrin is a beta-cyclodextrin, preferably “native.”

6. The cosmetic or dermatological composition according to claim 1, wherein it forms a skincare product, a hair treatment, an oral-dental treatment, a make-up, or a hair-removing treatment.

7. A use, in a cosmetic or dermatological composition, of at least one cyclodextrin which is at least partially present in the form of solid particles dispersed in a physiologically acceptable medium, wherein the cyclodextrin particles have:
a volume average size d(4.3), measured by laser particle size analysis, of less than or equal to 20 μm, more preferentially less than or equal to 12 μm, and most preferentially less than or equal to 8, and
a size distribution by volume, measured by laser diffraction particle size analysis, of which the characteristic diameters d(10), d(50) and d(90) are such that:
a. The diameter d(10) is less than or equal to 5.0 μm, preferentially less than or equal to 2.5 μm,
b. And the diameter d(50) is less than or equal to 15.0 μm, preferentially less than or equal to 10.0 μm,
c. And the diameter d(90) is less than or equal to 30.0 μm, preferentially less than or equal to 25.0 μm
for improving the sensory properties of said composition, preferentially for conferring ease of spreading, and/or a soft, powdery, non-greasy feel, and/or for preventing pilling.

8. The use according to claim 7, wherein the solid cyclodextrin particles have a size distribution by volume, measured by laser diffraction particle size analysis, having a coefficient of variation of less than or equal to 100%, preferentially less than or equal to 90%, and most preferentially less than or equal to 73%.

9. The use according to claim 7, wherein the cyclodextrin is a native beta-cyclodextrin.

10. The use according to claim 7, wherein the cosmetic or dermatological compositions are selected from a skincare product, a hair treatment, an oral-dental treatment, make-up, or a hair-removing treatment.

11. The use according to claim 10, wherein the cosmetic compositions are in the form of gels, emulsions, suspensions, lotions, serums, aerosols, mousses, pastes, powders or sticks, and preferentially of emulsions.

12. A method for preparing a cosmetic or dermatological composition, preferably an oil-in-water emulsion, comprising the following steps:
a. providing a physiologically acceptable medium,
b. The dispersion, in said physiologically acceptable medium, of at least one cyclodextrin having a volume average size d(4.3), measured by laser particle size analysis, of less than or equal to 20 μm, more preferentially less than or equal to 12 μm, and most preferentially less than or equal to 8, and a size distribution by volume, measured by laser diffraction particle size analysis, of which the characteristic diameters d(10), d(50) and d(90) are such that:
a. The diameter d(10) is less than or equal to 5.0 μm, preferentially less than or equal to 2.5 μm,
b. And the diameter d(50) is less than or equal to 15.0 μm, preferentially less than or equal to 10.0 μm,
c. And the diameter d(90) is less than or equal to 30.0 μm, preferentially less than or equal to 25.0 μm.

13. The method according to claim 12, wherein the content of cyclodextrin in the medium is 5% greater than the solubility thereof in said physiologically acceptable medium, preferably 10% greater than the solubility thereof, more preferentially 25% greater than the solubility thereof, and more preferentially 50% greater than the solubility thereof.
